(19) United States
(12) Patent Application Publication
(10) Pub. No.: US 2020/0323770 A1
(43) Pub. Date: Oct. 15, 2020

(54) WOUND DEBRIDEMENT COMPOSITION AND METHOD FOR TREATING WOUNDS

(71) Applicant: Hollister Incorporated, Libertyville, IL (US)

(72) Inventors: Abram D. Janis, Grayslake, IL (US); Brian E. Luedtke, North Barrington, IL (US)

(21) Appl. No.: 16/866,160

(22) PCT Filed: Oct. 31, 2018
(86) PCT No.: PCT/US2018/058503

§ 371 Date: May 4, 2020

Related U.S. Application Data
(60) Provisional application No. 62/592,855, filed on Nov. 30, 2017.

(51) Int. Cl. A61K 9/00; A61K 9/06; A61K 9/70; A61K 9/08; A61K 9/14

(52) U.S. Cl. CPC A61K 9/0014; A61K 9/06; A61K 45/06; A61K 9/08; A61K 9/14; A61K 9/70

(57) ABSTRACT

A combination of single or cocktails of analgesic, anesthetic, steroid and/or anti-epileptic drug or drugs with a topically applied or injected debridement agent may be used for wounds, burns, scars, Dupuytren's contracture and/or Peyronie's disease to allow for greater removal or breakdown of necrotic/devitalized, infected and/or fibriotic tissue through use of higher agent dose within a greater range of tolerable discomfort.

### BACKGROUND

The present disclosure relates generally to wound care and, more particularly, to a composition and method for reducing pain and discomfort associated with wound and burn debridement and the treatment of fibrosis, thereby extending the dose of the active agent and/or duration of therapy.

Skin, as the largest organ in the body, is a barrier separating the internal from the external, providing protection against infectious agents, mechanical or chemical trauma and other external insults, such as radiation. Given that skin faces injury and insults of varying scales on a daily basis, adaptation has allowed injuries to the skin in otherwise healthy individuals and within thresholds of damage to heal without intervention. However, for certain types of injuries, such as second and third degree burns, large traumatic wounds or chronic wounds in individuals with venous insufficiency, diabetes mellitus, decubitus ulcers or advanced age, the healing of the damaged tissue may be impaired by the presence of dead (or necrotic) tissue.

The removal of the dead and/or damaged tissue is widely accepted in the art of burn, trauma and chronic wound care to be necessary for the injury to heal and prevent exacerbation or future complications. See Zacharevskij, E., et al., Debridement method optimisation for treatment of deep dermal burns of the forearm and hand, EWMA J, 2017. 17(1): p. 7-13; Haury, B., et al., Debridement: an essential component of traumatic wound care, Am J Surg, 1978. 135(2): p. 238-42; Schultz, G. S., et al., Wound bed preparation: a systematic approach to wound management. Wound Repair Regen, 2003. 11 Suppl 1: p. S1-28. Standard second and third degree burn, and trauma treatment includes the debridement of non-viable tissue prior to autografting or primary closure, however debridement of burn wounds can be particularly painful even following premedication with opioids. See Sheridan, R. L., et al., Initial experience with a composite autologous skin substitute. Burns, 2001. 27: p. 421-424; Haury, B., et al., Debridement: an essential component of traumatic wound care; Lillieborg, S. and L. Aanderud, EMLA anaesthetic cream for debridement of burns: a study of plasma concentrations of lidocaine and prilocaine and a review of the literature, Int J Burn Trauma, 2017. 7(6): p. 88-97. It is well known that the dosage of opioids required for analgesia is increased in burn patients, in part due to the evolution of tolerance during repeated surgical and debridement procedures. See Bittner, E. A., et al., Acute and perioperative care of the burn-injured patient, Anesthesiology, 2015. 122(2): p. 448-464; Griggs, C., et al., Sedation and Pain Management in Burn Patients, Clin Plast Surg, 2017. 44(3): p. 535-540. It is also well known that delivering drugs, in this case analgesics, to the site of injury allows greater dosage without the associated risks from systemic exposure.

Accordingly, debridement, or the removal of non-living (necrotic) tissue or slough from a wound or burn, has long been considered the gold standard therapy in trauma and burn surgery as well as advanced wound care where it is the foundation of wound bed preparation. Debridement has been hypothesized to improve chronic wound healing by correcting the wound microenvironment to an acute wound trajectory by removing the local factors that have caused the wound to stall. The underlying pathophysiology of chronic wounds generates necrotic tissue that typically requires repeated debridement. See Schultz, G. S., et al., Wound bed preparation: a systematic approach to wound management. In particular, a direct correlation has long been known to exist between acceleration of wound healing and the extent of surgical debridement. See Steed, D. L., et al., Effect of extensive debridement and treatment on the healing of diabetic foot ulcers, Diabetic Ulcer Study Group, J Am Coll Surg, 1996. 183(1): p. 61-4, and Wilcox J. R., et al., Frequency of debridements and time to heal: A retrospective cohort study of 312,744 wounds. JAMA Dermatol, 2013. 149(9):1050-1058.

Necrotic tissue and slough are pro-inflammatory stimuli in the wound, are a nidus for colonization, infection and subsequent biofilm formation, and may impede contraction and reepithelialization by “splinting or stenting the wound”. See Schultz, G. S., et al., Wound bed preparation: a systematic approach to wound management; Ennis, W. J. and D. Hill, Wound Healing: A Comprehensive Assessment and Treatment Approach, in Skin Tissue Engineering and Regenerative Medicine, M. Z. Albanna and J. H. Holmes, Editors. 2016, Elsevier. Prolonged inflammation due to the presence of biofilm and other inflammatory stimuli can lead to elevated proteolytic activity that involves extracellular matrix (ECM) breakdown, growth factor and other cytokine degradation and cellular senescence that are characteristics of chronic dermal wounds in patients with diabetic, venous stasis and decubitus ulcers. See Steed, D. L., et al., Effect of extensive debridement and treatment on the healing of diabetic foot ulcers. Haury and colleagues demonstrated that devitalized tissue can serve as a culture medium for bacterial growth and inhibit the activity of leukocytes as well as create anaerobic conditions that can lead to particularly difficult to treat infections. See Haury, B., et al., Debridement: an essential component of traumatic wound care. It has also been postulated that non-debrided tissue can prevent the diagnosis of infection, abscess or wound depth by obscuration. See Schultz, G. S., et al., Wound bed preparation: a systematic approach to wound management; Steed, D. L., et al., Effect of extensive debridement and treatment on the healing of diabetic foot ulcers.

Surgical and sharp debridement include excision with a scalpel, scissors, curette or other tool for the removal of necrotic tissue, often with anesthesia. Medical practice guidelines, however, limit the performance of surgical/sharp debridement to physicians, podiatrists and certain advanced nursing specialties. This constraint has encouraged the rapid growth of alternative debridement technologies and techniques. For example, these technologies and techniques may include 1) autolytic debridement, where moist or moisture containing dressings are used to increase the activity of endogenous proteolytic enzymes within the wound bed, 2) mechanical debridement, which includes the use of ultrasound, saline sprays, whirlpools and fibrous swabs, 3) biosurgical debridement, which uses Lucilia sericata, or green bottle fly larvae (maggots), 4) enzymatic debridement, which uses proteases, or enzymes that break down proteins, and 5) chemical debridement, which involves the use of protein denaturing chemicals and detergents, including biosurfactants.

Chemical debridement agents include but are not limited to urea, hydrogen peroxide, and ionic, non-ionic, and amphoteric surfactants such as polysorbate, tween 80, sodium dodecylsulfate (SDS), Triton X-100, and poloxamer, for example pluronic F68. See Percival, S. L. et al. Surfactants and their role in wound cleansing and biofilm management. J Wound Care 2017. 26(11):680-690. Biosurfactants are amphiphilic molecules produced by microorganisms, including but not limited to lipopeptides (such as polymixins, putisolvin, pseudofactin, and surfactin), and glycolipids (such as rhamnolipids from Pseudomonas aeruginosa and Sophorolipids from the yeast genus Candida). See Banat, I. M. et al., Microbial biofilms: Biosurfactants as antibiofilm agents. Appl Microbiol Biotechnol 2014. 98(24):9915-9929.

As necrotic tissue can be anchored to the wound bed by collagen fibrils, collagenase has been used for decades to release the eschar and enable the formation of granulation tissue, which is necessary for reepithelialization to occur. Currently collagenase from the bacterium Clostridium histolyticum is the only enzymatic debridement agent marketed in the United States and Canada. A bromelain based enzymatic debridement agent is marketed in Europe. Bromelain has been described as causing pain to patients in the literature. Collagenase's current labeling includes reference to transient erythema, or redness. Papain-urea, which was removed from the market in the 1990s, has also been reported to cause pain during application. Other enzymes historically used as debridement agents include but are not limited to fibrinolysin, fibrinolysin/deoxyribonuclease, trypsin, papain, chymotrypsin from Lucilia sericata, and proteases from the digestive system of Antarctic krill (Euphausia superba). See Mekkes J. R., et al. Efficient debridement of necrotic wounds using proteolytic enzymes derived from Antarctic krill: a double-blind placebo-controlled study in a standardized animal wound model. Wound Repair Regen 1998. 6:50-58.

Chemical debridement agents have historically included Dakin's solution, hydrogen peroxide, Iodosorb, Oakin, and Mesalt. With both enzymatic and chemical debridement, it is known that increasing the dose of the active ingredient can increase the effectiveness of the therapy. An increase in the dose of the active ingredient, however, is likely to cause more redness, irritation, discomfort and/or pain at the wound site. See Shi, L., et al., Study on the debridement efficacy of formulated enzymatic wound debriding agents by in vitro assessment using artificial wound eschar and by an in vivo pig model, Wound Repair Regen, 2009. 17(6): p. 853-62. This correlation has, therefore, effectively limited the dose of active ingredient utilized and/or duration of therapy, which has, in turn, limited the effectiveness of these therapies and their promotion of improved wound, burn and scar healing.

For example, Mekkes et al. demonstrated a dose response for krill-derived digestive enzymes ranging from 0.6 U/mL to 6.0 U/mL. See Mekkes J. R., et al. Efficient debridement of necrotic wounds using proteolytic enzymes derived from Antarctic krill: a double-blind placebo-controlled study in a standardized animal wound model. Wound Repair Regen 1998. 6:50-58. Shi et al. showed that increasing papain concentrations ranging from 200-1,600 USP units/mg in 10% urea correlated with increased fibrinolysis, collagenolysis, and elastinolysis in vitro, yet treatment of wound in vivo with concentrations of 400, 800 and 1,600 U/mg resulted in greater erythema than 200 U/mg. See Shi, L., et al., Study on the debridement efficacy of formulated enzymatic wound debriding agents by in vitro assessment using artificial wound eschar and by an in vivo pig model, Wound Repair Regen, 2009. 17(6): p. 853-62.

In addition to use in chronic wounds and burns, the use of debridement agents (for example, collagenase) has been described for the treatment of fibrosis in plastic and reconstructive surgery, including the treatment of Peyronie's disease, Dupuytren's contracture, hypertrophic and keloid scars, and scar release. It should be noted that in these applications for fibrotic diseases, the agent is typically injected to an internal site.

In view of the above, there is a need for debridement formulations that expand the tolerable dose range of the active ingredient to facilitate debridement while minimizing redness, irritation, discomfort and/or pain, which may also allow for decrease in the length of time between debridements, thereby decreasing the duration and/or extent of treatment.

### BRIEF SUMMARY

It is an object of the present disclosure to provide a wound debridement composition.

It is another object of the present disclosure to provide a wound debridement composition with analgesic properties.

It is another object of the present disclosure to provide a wound debridement composition that effectively increases the tolerable dose range of active ingredient to facilitate wound healing. The wound debridement composition of the present disclosure may include both topically applied, as a solution, gel, beads, powder or as a coating on or component of a dressing or silicone sheet, as well as injected compositions. For example, in fibrotic disease applications, the debridement composition may be injected to an internal site.

Wound debridement compositions of the present disclosure uses a combination of single or cocktails of analgesic, anesthetic, steroid and/or anti-epileptic drug or drugs with a topically applied or injected debridement agent for acute and chronic wounds, burns, scars, Dupuytren's contracture and/or Peyronie's disease and other fibrotic disorders to allow for greater removal or breakdown of necrotic/devitalized, infected and/or fibrotic tissue through the use of higher agent dose within a greater range of tolerable discomfort.

In one aspect, a wound debridement composition having analgesic properties is provided. The composition may comprise at least one debridement agent and at least one ingredient having analgesic properties.

In an embodiment, the at least one debridement agent may be selected from a group consisting of urea, proteases, collagenases, papain, bromelain, fibrolysin trypsin, chymotrypsin, surfactants (synthetic & biosurfactants), detergents, Dakin's solution, iodine solution, hyper salt solutions, hypotonic salt solutions, saline solutions, and water.

The at least one ingredient having analgesic properties may be selected from a group consisting of non-steroidal anti-inflammatory drugs, cyclooxygenase-2 selective inhibitors, salicylates, steroids, opioids, anesthetics, and naturally derived and synthetic soothing agents with anti-inflammatory properties. For example, the at least one ingredient having analgesic properties may be selected from a group consisting of ibuprofen, naproxen, ketoprofen, piroxicam, indomethacin, diclofenac, nimesulide, celecoxib, valdecoxib, acetyl salicylic acid, cortisone, prednisone, morphine, fentanyl, lidocaine, prilocaine, bupivacaine, benzocaine, tetracaine, dibucaine, paracetamol, acetaminophen, evening primrose oil, borage seed oil, black currant seed oil, cannabinoids, felbinac, nicotinate esters, capsaicin, arnitriptyline, glyceryl trinitrate, menthol, camphor, pimecrolimus, phenytoin, benzyl alcohol, ethyl chloride, hexylresorcinol, trolarnine, pramoxine, proparacaine, almond oil, aloe vera, castor oil, eucalyptus oil, grapeseed oil, menthol, peppermint oil, sandalwood oil, tea tree oil, and tumeric. The at least one ingredient having analgesic properties may be included in an amount to expand a tolerable dose range of the at least one debridement agent.

In any of the foregoing embodiments, the wound debridement composition may be configured for a topical application or configured to be injected into an internal site.

Other aspects, objectives and advantages will become more apparent from the following detailed description.

### DETAILED DESCRIPTION

The present disclosure is directed to a wound debridement composition that reduces or prevents pain or discomfort associated with the agent, mode of action, the debridement process, and/or existing pain from a wound or burn. By reducing the amount of pain resulting from the agent's mode of action or side effect, the debridement process or the pain inherent in a wound, burn, or fibrotic tissue, the tolerable concentration or dose of the active ingredient may be increased, thereby resulting in improved debridement therapy and overall effectiveness.

In an embodiment, the composition uses a topical or injected debridement agent in combination with an analgesic or anesthetic. Suitable analgesics or anesthetics may include, but are not limited to, non-steroidal anti-inflammatory drugs (e.g., ibuprofen, naproxen, ketoprofen, piroxicam, indomethacin, diclofenac), cyclooxygenase-2 selective inhibitors (e.g., nimesulide, celecoxib, valdecoxib), salicylates (e.g., acetyl salicylic acid), steroids (e.g., cortisone, prednisone) and opioids (e.g., morphine, fentanyl). Suitable anesthetics may include, but are not limited to, lidocaine, prilocaine, bupivacaine, benzocaine, tetracaine, and dibucaine. In addition, other possible analgesics/anesthetics may include, but are not limited to, paracetamol (acetaminophen), evening primrose oil, borage seed oil, black currant seed oil, cannabinoids, felbinac, nicotinate esters, capsaicin, arnitriptyline, glyceryl trinitrate, menthol, camphor, pimecrolimus, phenytoin, benzyl alcohol, ethyl chloride, hexylresorcinol, trolarnine, pramoxine, and proparacaine. Naturally derived and synthetic soothing agents with anti-inflammatory properties including, but not limited to, almond oil, aloe vera, castor oil, eucalyptus oil, grapeseed oil, menthol, peppermint oil, sandalwood oil, tea tree oil, and/or tumeric may be combined with the debridement agent as well.

Suitable topical or injected debridement agents may include, but are not limited to, urea, proteases (e.g., collagenases, papain, bromelain, fibrolysin trypsin and chymotrypsin), chemical debridement agents such as surfactants (e.g., detergents), Dakin's solution, iodine solution, hyper/hypotonic salt solutions and/or solutions used in mechanical debridement (e.g., saline, water for injection).

The present disclosure therefore discloses the inclusion of a drug or drugs to a topical or injected debridement formulation to reduce or prevent pain associated with the agent mode of action, the debridement process, or existing pain from a wound or burn, which allows the tolerable concentration or dose of the active ingredient to be increased. The combination of a debridement agent with additional analgesic compounds may provide a composition that expands the armamentarium of wound care clinicians, burn surgeons and plastic reconstructive surgeons alike, and spares patients from the pain and discomfort typically associated with wound bed preparations.

The wound debridement composition may be provided in the form of a solution, gel, beads, powder and the like. In some embodiments, the wound debridement composition may be configured for topical applications. For example, the wound debridement composition may be provided as a coating on a substrate, such as a dressing, silicone sheet, gel, and the like, and/or incorporated into the substrate. In other embodiments, the wound debridement composition may be configured to be injected to an internal site.

While the present disclosure discloses the use of enzymatic, chemical and mechanical debriding agents with analgesics to expand the tolerable dose range of the active ingredient of the debriding agent, the present disclosure is not so limited in this regard. In particular, it is also contemplated that analgesics and/or anesthetics can be used in combination with autolytic and/or biosurgical agents, dressings and processes to minimize or eliminate patient pain, discomfort and irritation associated with such agents, dressings and processes to maximize debridement and healing. Incorporation of analgesics may also allow for a decrease in the length of time between debridement procedures, thereby increasing efficacy of therapy and decreasing the overall duration of treatment.

All patents referred to herein, are hereby incorporated herein in their entirety, by reference, whether or not specifically indicated as such within the text of this disclosure.

In the present disclosure, the words “a” or “an” are to be taken to include both the singular and the plural. Conversely, any reference to plural items shall, where appropriate, include the singular.

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

1. A wound debridement composition having analgesic properties, the composition comprising at least one debridement agent and at least one ingredient having analgesic properties.

2. The wound debridement composition of claim 1, wherein the at least one debridement agent is selected from the group consisting of urea, proteases, collagenases, papain, bromelain, fibrolysin trypsin, chymotrypsin, surfactants, detergents, Dakin's solution, iodine solution, hyper salt solutions, hypotonic salt solutions, saline solutions, water, and mixtures thereof.

3. The wound debridement composition of claim 1, wherein the at least one ingredient having analgesic properties is selected from the group consisting of non-steroidal anti-inflammatory drugs, cyclooxygenase-2 selective inhibitors, salicylates, steroids, opioids, anesthetics, naturally derived and synthetic soothing agents with anti-inflammatory properties, and mixtures thereof

4. The wound debridement composition of claim 1, wherein the at least one ingredient having analgesic properties is selected from a group consisting of ibuprofen, naproxen, ketoprofen, piroxicam, indomethacin, diclofenac, nimesulide, celecoxib, valdecoxib, acetyl salicylic acid, cortisone, prednisone, morphine, fentanyl, lidocaine, prilocaine, bupivacaine, benzocaine, tetracaine, dibucaine, paracetamol, acetaminophen, evening primrose oil, borage seed oil, black currant seed oil, cannabinoids, felbinac, nicotinate esters, capsaicin, arnitriptyline, glyceryl trinitrate, menthol, camphor, pimecrolimus, phenytoin, benzyl alcohol, ethyl chloride, hexylresorcinol, trolamine, pramoxine, proparacaine, almond oil, aloe vera, castor oil, eucalyptus oil, grapeseed oil, menthol, peppermint oil, sandalwood oil, tea tree oil, turmeric, and mixtures thereof.

5. The wound debridement composition of claim 1, wherein the at least one ingredient having analgesic properties is included in an amount to expand a tolerable dose range of the at least one debridement agent.

6. The wound debridement composition of claim 1, wherein the at least one ingredient having analgesic properties is included in an amount to increase effectiveness and/or rate of debridement and therefore decrease a length of time between debridement and to decrease a duration of treatment.

7. The wound debridement composition of claim 1, wherein the wound debridement composition is provided in the form of a solution, gel, beads, or powder.

8. The wound debridement composition of claim 1, wherein the composition is configured for a topical application.

9. The wound debridement composition of claim 8, wherein the wound debridement composition is provided as a coating on a substrate.

10. The wound debridement composition of claim 9, wherein the wound debridement composition is incorporated into a substrate.

11. The wound debridement composition of claim 9, wherein the substrate is a dressing, silicone sheet or gel.

12. The wound debridement composition of any of claim 1, wherein the composition is configured to be injected into an internal site.

13. The wound debridement composition of claim 10, wherein the substrate is a dressing, silicone sheet or gel.
